(19) United States
(12) Patent Application Publication
(10) Pub. No.: US 2006/0069336 A1
(43) Pub. Date: Mar. 30, 2006

(54) Ankle interface

(75) Inventors: Hermano Igo Krebs, Cambridge, MA (US); Neville Hogan, Sudbury, MA (US); Jason William Wheeler, Albuquerque, NM (US); Dustin Williams, Cambridge, MA (US)

Correspondence Address: FOLEY HOAG, LLP;PATENT GROUP, WORLD TRADE CENTER WEST, 155 SEAPORT BLVD, BOSTON, MA, 02110, US

(73) Assignee: Massachusetts Institute of Technology, Cambridge, MA (US)

(21) Appl. No.: 11/236,470

(22) Filed: Sep. 27, 2005

Related U.S. Application Data
(60) Provisional application No. 60/613,421, filed on Sep. 27, 2004.

(51) Int. Cl. A61F 5/00

(52) U.S. Cl. 602/28

(57) ABSTRACT

An ankle interface may include a leg connection attachable to a user's leg, a foot connection attached to the user's corresponding foot, and a transmission system coupling the leg connection and the foot connection with at least two degrees of freedom and actuating at least two degrees of freedom.

### CROSS-REFERENCE TO RELATED APPLICATION

This application claims the benefit of U.S. Provisional Application No. 60/613,421, filed Sep. 27, 2004, the contents of which are hereby incorporated herein by reference.

### BACKGROUND

Neurological trauma, orthopedic injury, and joint diseases are common medical problems in the United States. A person with one or more of these disorders may lose motor control of one or more body parts, depending on the location and severity of the injury. Recovery from motor loss frequently takes months or years, as the body repairs affected tissue or as the brain reorganizes itself. Physical therapy can improve the strength and accuracy of restored motor function and can also help stimulate brain reorganization. This physical therapy generally involves one-on-one attention from a therapist who assists and encourages the patient through a number of repetitive exercises. The repetitive nature of therapy makes it amenable to administration by properly designed robots.

Existing devices for physical therapy are by and large CPM (continuous passive motion) machines. CPM machines have very high mechanical impedance and simply move the patient passively through desired motions. These devices might be useful to extend the range of motion. However, because these systems do not allow for impedance variation, patients are not encouraged to express movement on their own. Support devices for the ankle and foot, called ankle-foot orthoses (AFOs), are also used. AFOs are entirely passive devices that can align the ankle and foot, suppress spastic motions, and support weak muscles. In so doing, they can actually diminish a user's ankle strength and motion because they chiefly constrain the ankle.

### SUMMARY

This disclosure describes robotic ankle interfaces that may support therapy by guiding, assisting, resisting, and/or perturbing ankle motion.

An ankle interface may include a leg connection attachable to a user's leg, a foot connection attachable to the user's corresponding foot, and a transmission system coupling the leg connection and the foot connection with at least two degrees of freedom and actuating at least two degrees of freedom.

A method of ankle training may include attaching a subject's leg and foot to the ankle interface, and actuating the transmission system to provide at least one of assistance, perturbation, and resistance to an ankle motion.

### BRIEF DESCRIPTION OF THE DRAWINGS

FIGS. 1A-C depict motions of the ankle and foot.

FIG. 2 shows an exemplary embodiment of an ankle interface.

FIGS. 3-8 depict various embodiments of kinematic mechanisms for ankle interfaces.

FIGS. 9-9J show embodiments of transmissions for ankle interfaces.

FIG. 10 shows a linkage diagram of the kinematic mechanism of FIG. 8.

FIGS. 11-11A show exemplary embodiments of leg connections.

FIG. 12 shows an exemplary embodiment of a foot connection.

FIG. 13 shows a photograph of the ankle interface of FIG. 12 attached to a user's leg and foot.

FIGS. 14A-D show kinematics of an ankle interface.

FIGS. 15 and 16 show additional embodiments of ankle interfaces.

FIGS. 17-21 show various views of another embodiment of an ankle interface.

FIG. 22 depicts a cross-sectional view of a portion of an ankle interface in relation to a shoe.

### DETAILED DESCRIPTION

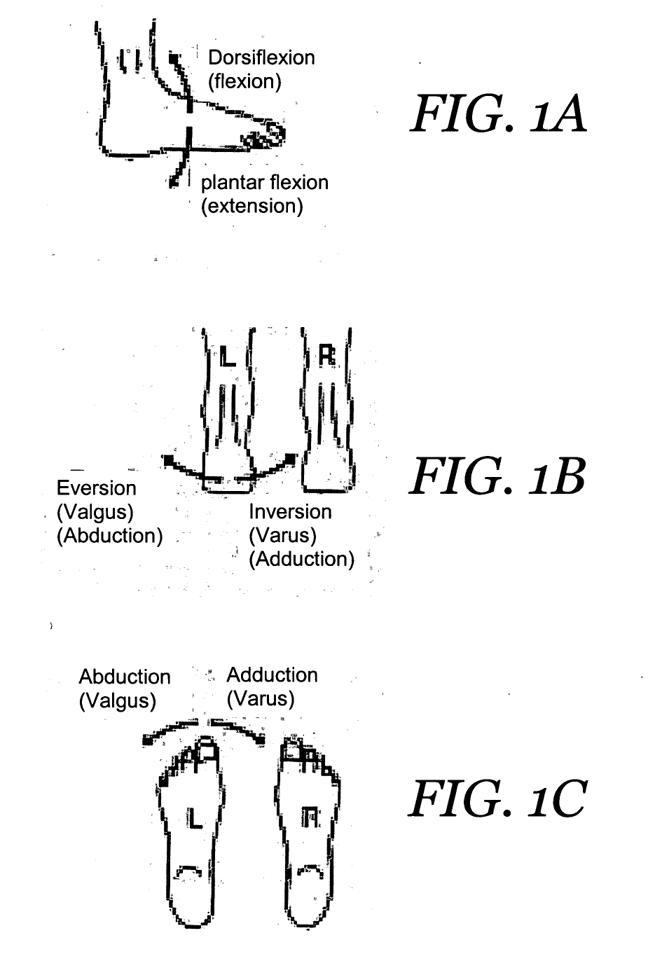

The ankle interfaces described herein can be used to provide physical therapy to a subject and/or measure motions of the ankle. The ankle is the joint that couples the leg and the foot. This joint is composed of a complex of bones, tendons, and ligaments. The joint permits motion with several degrees of freedom, including dorsiflexion/plantar flexion, in which the foot tilts up or down (FIG. 1A), and inversion/eversion, in which the foot tilts side-to-side (FIG. 1B). The foot can also sweep side-to-side, called adduction/abduction (FIG. 1C). This motion results largely from rotation of the leg, but the ankle may contribute some rotation to this motion. All three of these motions are important in normal gait, with dorsiflexion/plantar flexion being the most important of the three for gait.

In particular, the ankle interface may include attachment elements to connect the device to the user's leg and foot, a set of motors, and a transmission system (such as linkages) that can apply torques to an ankle about one or more axes of rotation. In some modes, an ankle interface can deliver assistance torques to a subject (i.e., torques that assist a subject in moving the ankle in the desired way). In other modes, an ankle interface can deliver resistance torques (i.e., torques that oppose a desired motion, as a way of building strength) and/or perturbation forces (i.e., forces directed at oblique angles to a subject's intended motion) to assess stability or neuro-muscular control.

A controller, such as a programmed computer, may direct the actuation of the transmission system to execute a rehabilitation or training program. An ankle interface can be combined with device for actuating other joints, such as at the knee, the hip, and/or the pelvis, in order to provide coordinated therapy for a subject's lower extremity. The disclosed systems can also be combined with other technologies, such as electromyography (EMG), electroencephalography (EEG) and various modes of brain imaging, and used to correlate ankle motion to muscle, nerve and brain activity and to study ankle movement control. These applications are described in greater detail below. In some embodiments, the ankle interfaces described here are rotatable in one, two, or more degrees of freedom. In some instances, an ankle interface is exoskeletal—i.e., the device is built around the user. In others, the interface may be non-exoskeletal.

Ankle interfaces can use impedance control to guide a subject gently through desired movements. If a patient is incapable of movement, the controller can produce a high impedance (high stiffness) between the desired position and the patient position to move the patient through a given motion. When the user begins to recover, this impedance can gradually be lowered to allow the patient to create his or her own movements. An ankle interface can also be made mechanically backdrivable. That is, when an interface is used in a passive mode (i.e. no input power from the actuators), the impedance due to the mechanical hardware (the effective friction and inertia that the user feels when moving) is small enough that the user can easily push the attachment around.

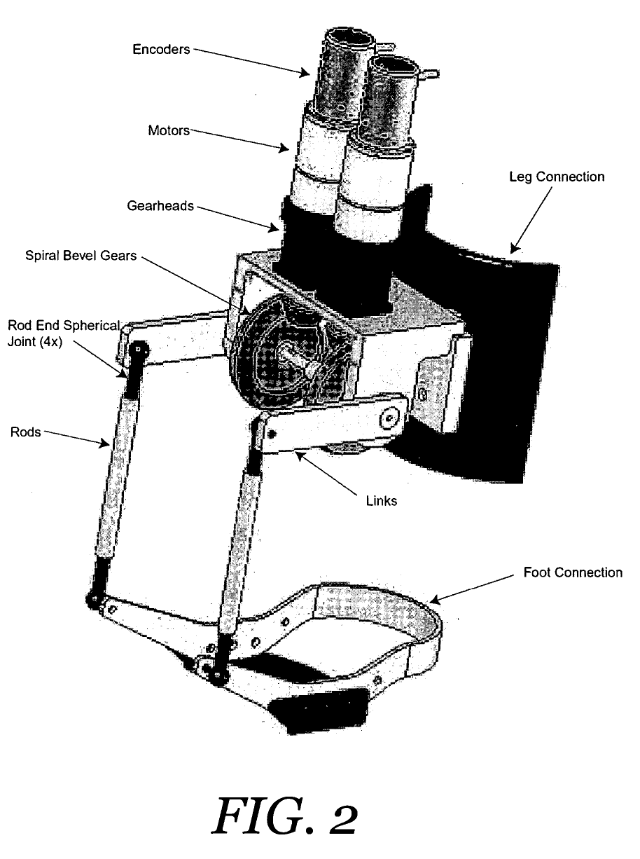

FIG. 2 shows an exemplary embodiment of an ankle interface. The device includes a leg connection that attaches the interface to a user's leg. The leg connection may include one or more straps that extend around the user's leg to hold the device against the leg. The leg connection may include a knee-brace to help immobilize the device with respect to the knee and prevent motion of the device relative to the leg. The interface may also include a foot connection that receives the foot. The leg connection and the foot connection may be coupled to one another through a motor and transmission system. The motor and transmission system can develop forces to move the foot relative to the leg in various motions, such as dorsiflexion/plantar flexion and inversion/eversion. In the exemplary embodiment shown in FIG. 2, the motor and transmission system includes two motors coupled to respective gear systems. The gears drive a series of links and joints that are attached to the foot connection. The transmission system can also include one or more sensors that can detect the rotation state of the device. In the depicted example, the sensors are encoders that detect the rotational displacement and angular velocity of the respective motors, as well as force and torque sensors.

In some embodiments, an ankle interface allows normal range of motion in all three degrees of freedom of the foot relative to the shank (lower leg) during walking. Specifically, it can allow 25° dorsiflexion, 45° plantar flexion, 25° inversion, 15° eversion, and 15° of adduction or abduction. These ranges are near the limits of range of comfortable motion for normal subjects and beyond what is required for typical gait. In some embodiments, an ankle interface can provide independent, active assistance, resistance, or perturbation in two of these three degrees-of-freedom, namely, dorsi/plantar flexion and inversion/eversion, and a passive degree-of-freedom for adduction/abduction. There is an additional advantage of actuating fewer degrees of freedom than are anatomically present: it allows the device to be installed without precise alignment with the patient's joint axes (ankle and subtalar joints) causing excessive forces or torques or compromising the functioning of the device. Some embodiments, however, can actuate adduction/abduction.

The motor and transmission system will typically include one or more actuators coupled through a series of linkages to the user's foot and/or leg. The motor and transmission system can deliver forces to the ankle and/or leg that result in torques at the ankle. The applied torques can act on the dorsiflexion/plantar flexion motion, the inversion/eversion motion, or both. The system can be configured to allow free adduction/abduction motion independent of the system, or can include an actuator that applies torques on this motion as well. In some embodiments, the system is designed to facilitate, perturb, or resist ankle motion with two degrees of freedom: dorsiflexion/plantar flexion and inversion/eversion.

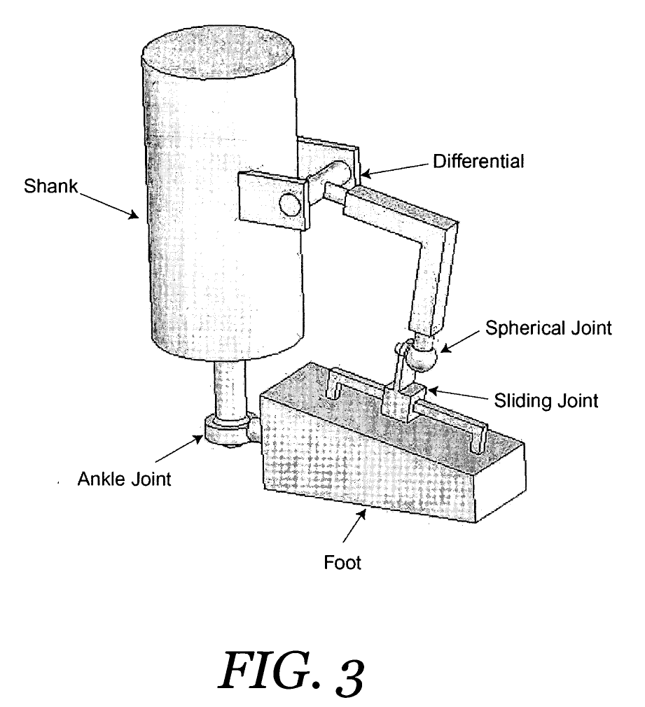

A wide variety of transmission systems are contemplated. Several are illustrated in FIGS. 3-8 as idealized kinematic mechanisms.

FIG. 3 shows a kinematic mechanism that includes a differential attached to the user's leg (shank) and a sliding joint on the foot. They are connected by a two links and a spherical joint.

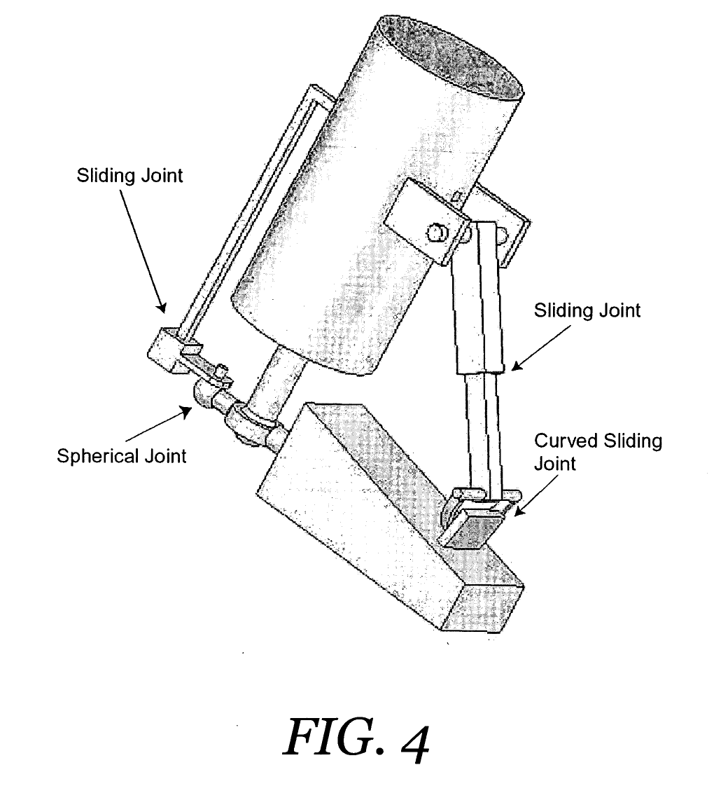

FIG. 4 depicts another kinematic mechanism. This mechanism includes three sliding joints. One is placed behind the leg and would be actuated to provide dorsi/plantar flexion moments. It is connected to the heel with a spherical joint. The other two sliding joints are in front of the leg and would provide moments for inversion and eversion. The sliding joint on the foot has a curved rail to allow rotation about the foot axis.

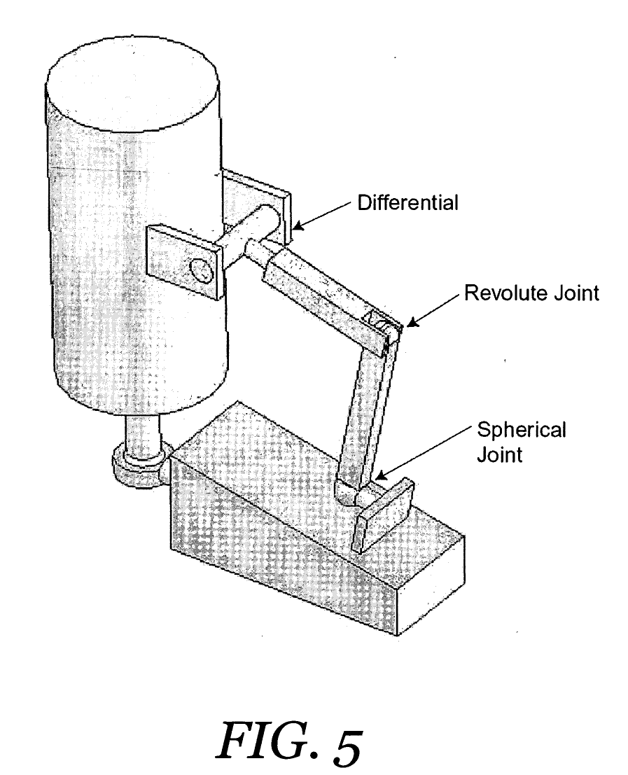

FIG. 5 depicts yet another kinematic mechanism. This mechanism includes a two-link serial mechanism connected to the shank with a differential and to the foot with a spherical joint. The primary moments will be produced in the dorsi/plantar flexion and adduction/abduction directions.

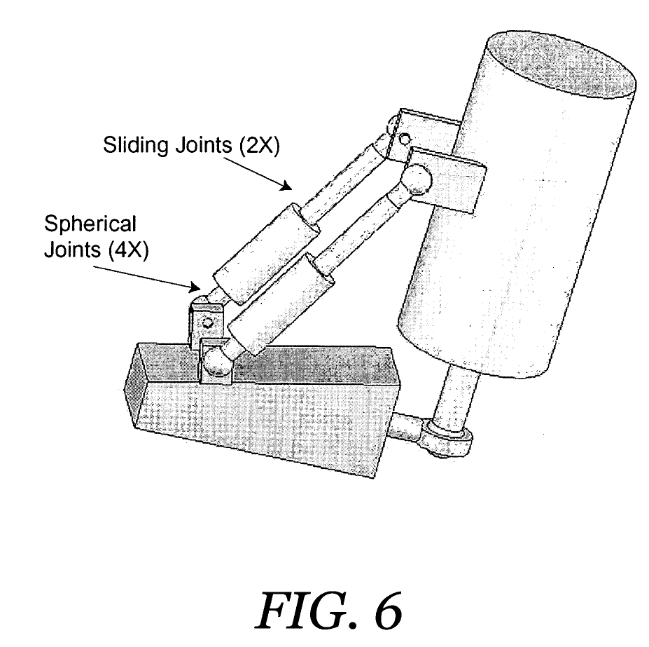

FIG. 6 depicts another kinematic mechanism which includes two sliding joints or actuators mounted in parallel with spherical joints on either end. This mechanism will allow actuation in dorsiflexion/plantar flexion and inversion/eversion.

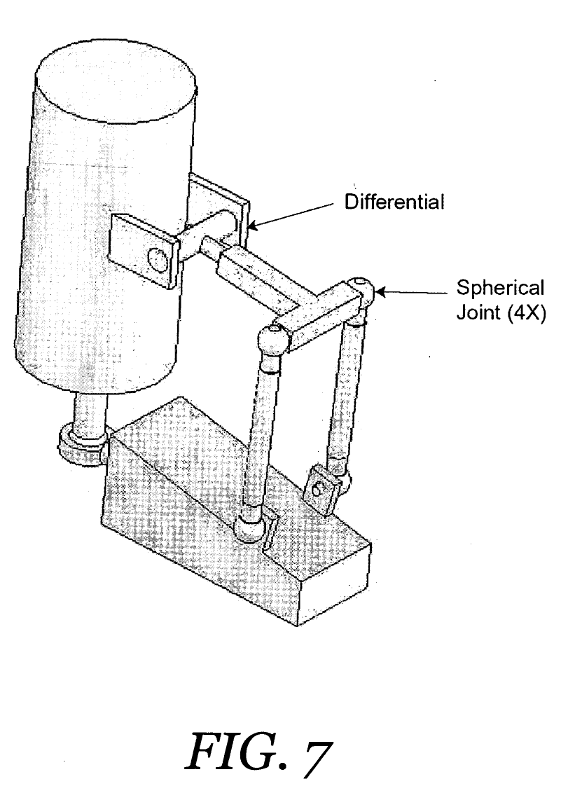

FIG. 7 shows still another kinematic mechanism which includes a single link mounted between a differential and two rods that connect to the foot. Spherical joints are mounted at either end of these rods. This mechanism will allow actuation in dorsiflexion/plantar flexion and inversion/eversion.

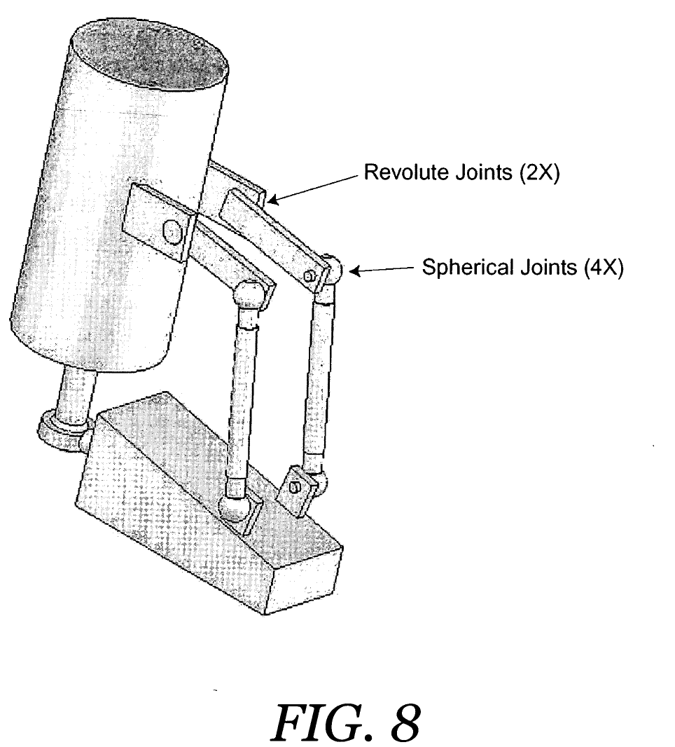

FIG. 8 shows another kinematic mechanism which is a modification of the mechanism shown in FIG. 7. The main link was converted to two links, each with a single degree of freedom, by essentially turning the differential “inside out” to create two independent revolute joints. Motion is produced by actuating the links on the shank. If both links move in the same direction, a moment is created at the ankle to produce dorsi/plantar flexion. If the links move in opposite directions, the resulting moment produces inversion/eversion. Combinations of these movements is also possible. Locating the patient axes is not required using this approach. The rods produce forces on the foot which project to the patient axes.

The mobility, M, of many linkages can be determined using Gruebler's mobility equation, which can be expressed as

$$M = 6 ( n - j - 1 ) + {\sum }_{i = 1}^{j} {f}_{i}$$

where n is the number of links, j is the number of joints and f is the mobility provided by joint i. If the ankle is modeled as a single joint with a mobility of 3 and the foot and shank as rigid links, the desired mobility of the system with the ankle interface attached is 3. Whether this model is physiologically accurate is unimportant. For design purposes, the robot/patient system must only have the same mobility as the model of the ankle and foot. The FIG. 8 mechanism includes two serial 2-link mechanisms mounted in parallel. The links that connect to the foot are mounted with spherical joints on either end. The links attached to the shank have only a single degree of freedom. For this system, Gruebler's equation actually predicts a mobility of 5. However, two of these degrees of freedom are the rotations of the links connecting to the foot and have no effect on the movement of the foot relative to the shank. Disregarding these benign degrees of freedom, the chosen configuration has the desired mobility of 3.

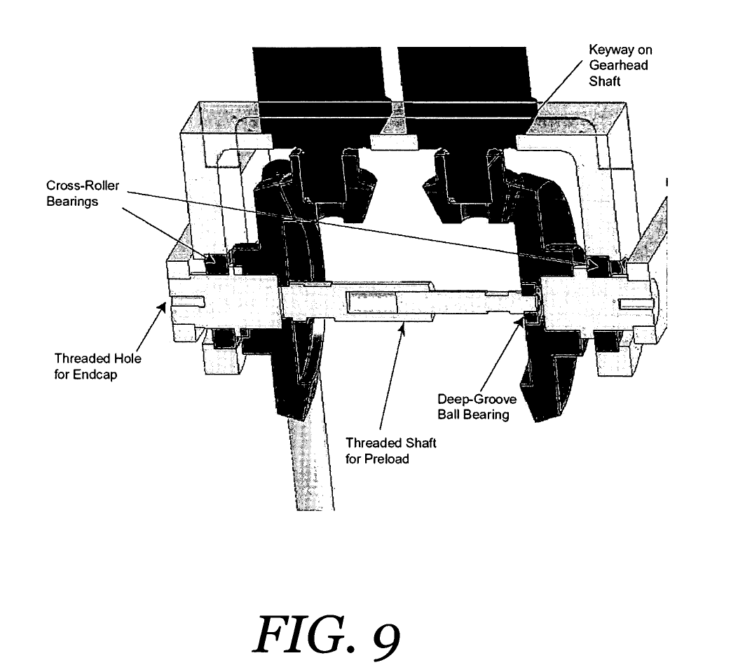

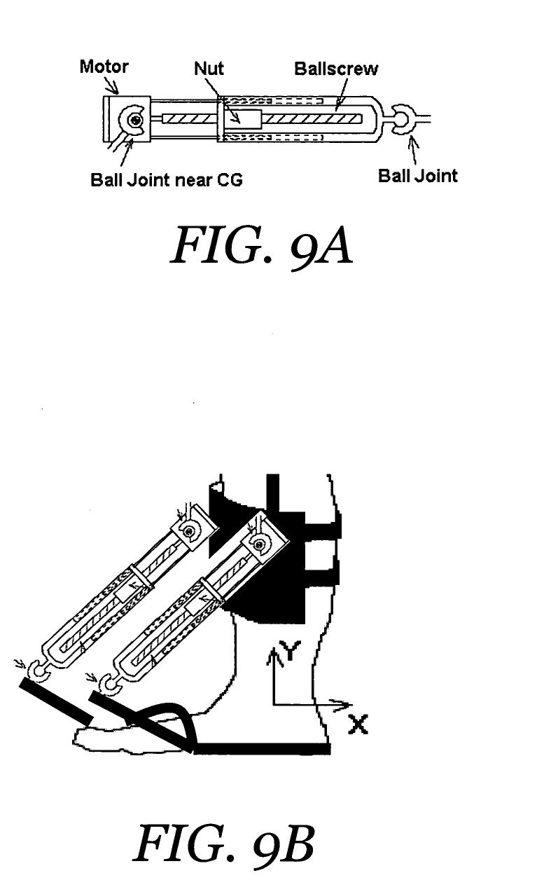

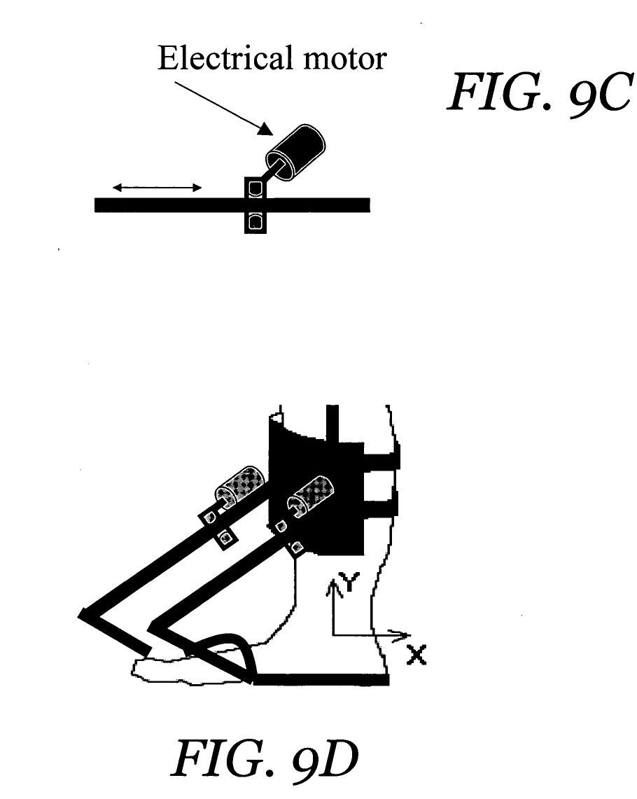

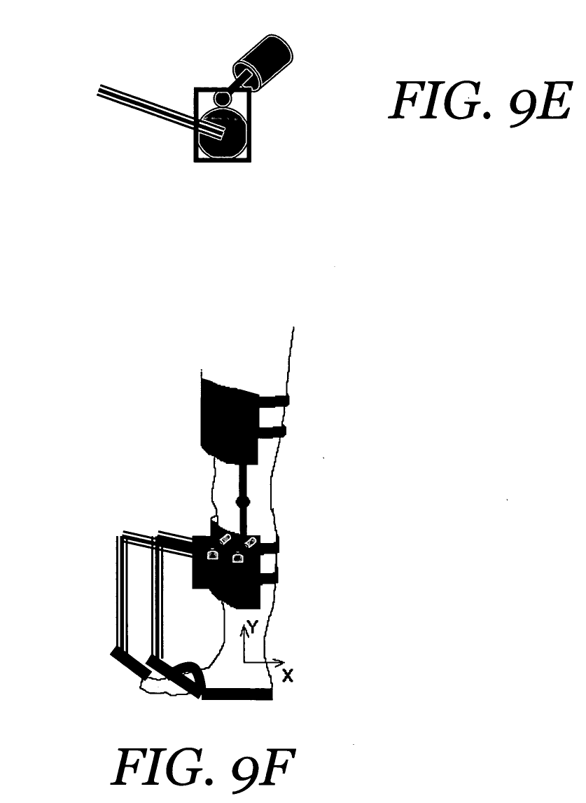

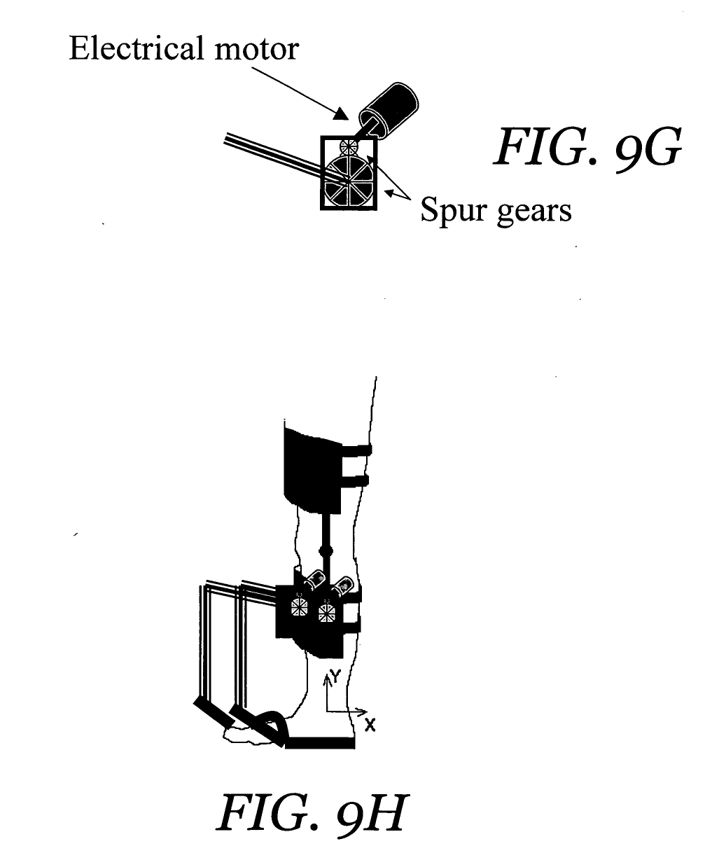

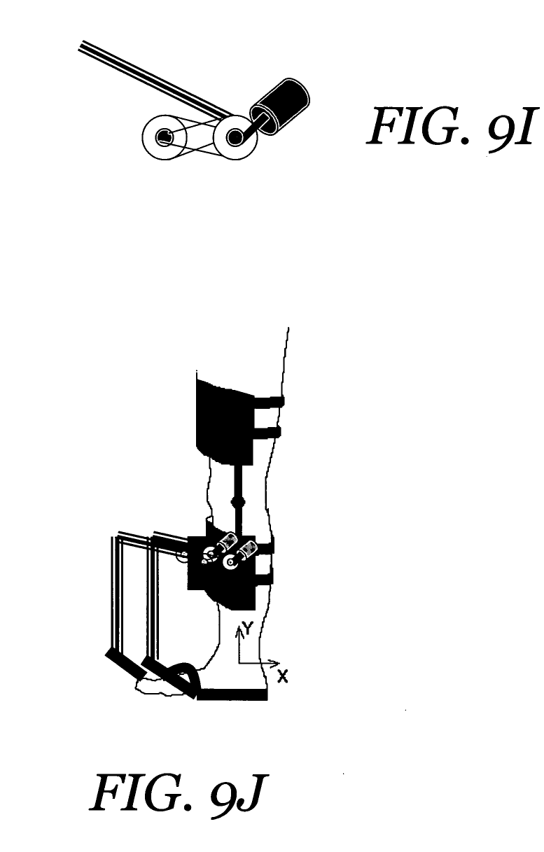

FIG. 9 shows detail of one embodiment of a gear system that can be used with the kinematic mechanism shown in FIG. 8. The gear system transmits torques from the actuators to the linkages operating on the foot. In this embodiment, each motor couples through a series of gears to a respective link. In some embodiments, the actuators should be selected, and the transmission system arranged, so that the device can assist hypertonic patients. In this case, the system can deliver 17 N·m in each actuated degree of freedom.

FIGS. 9A-9J show several other exemplary embodiments of transmission systems. FIG. 9A shows a linear ball screw actuator. Two linear actuators can be used, as in the kinematic mechanism of FIG. 6. A schematic of the resulting ankle interface is shown in FIG. 9B. Other linear actuators can be used, such as a standard lead screw. A strain gauge may be placed on the screw, between the nut and the motor, as a force sensor.

FIG. 9C shows a linear friction (or traction) drive actuator. Two linear friction drive actuators can be used in parallel, as shown in FIG. 9D. Polyurethane wheels, for example, can be used; they can easily be replaced if they wear. The forces on the motor shaft in this embodiment and other transmission shafts can be high. This can be alleviated by using a second wheel on the opposite side which balances the radial force on the shaft.

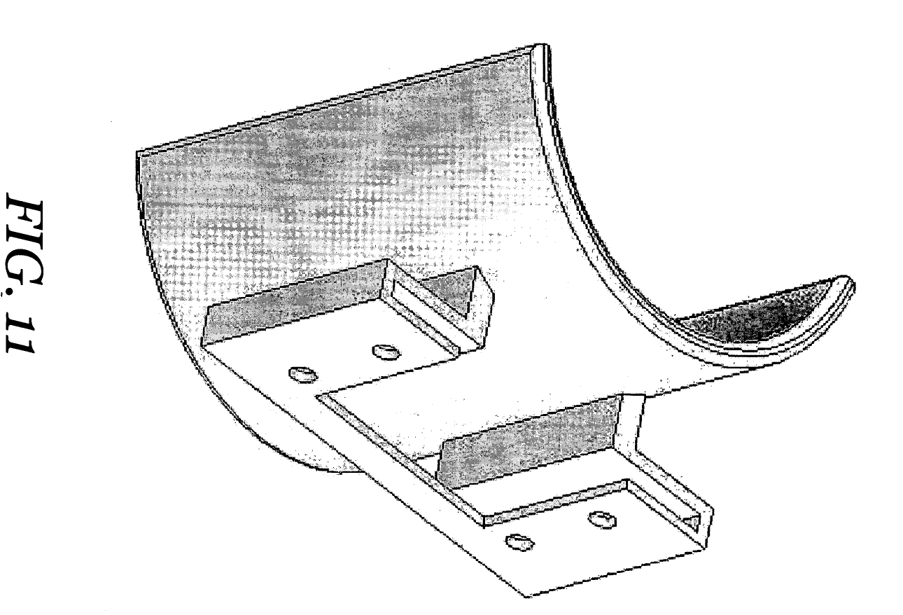

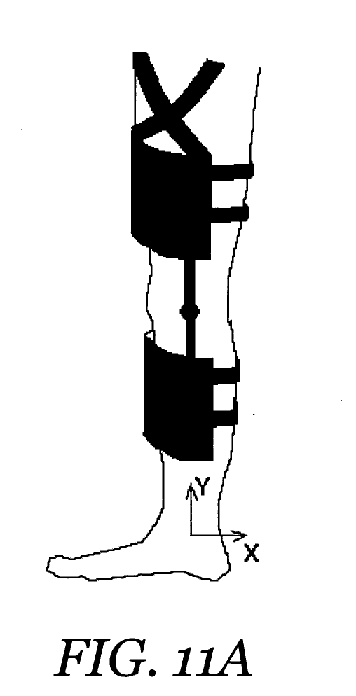

FIG. 9E shows a rotary friction drive actuator. Two rotary friction drive actuators can be used in parallel, as shown in FIG. 9F. The depicted interface includes an alternative leg component, shown in FIG. 11A. In some embodiments, the motors can be placed behind the calf to counterbalance the weight.

FIG. 9G shows a rotary gear drive actuator. Two rotary gear drive actuators can be used in parallel, as shown in FIG. 9H.

FIG. 9I shows a cable drive actuator. The cable drive actuator can include two pulleys. In some embodiments, the motors can be placed behind the calf to counterbalance the weight. An exemplary ankle interface with a cable drive actuator is shown in FIG. 9I.

An actuator may be a combination of the actuators described above. For example, an actuator may be both a traction drive and a screw drive.

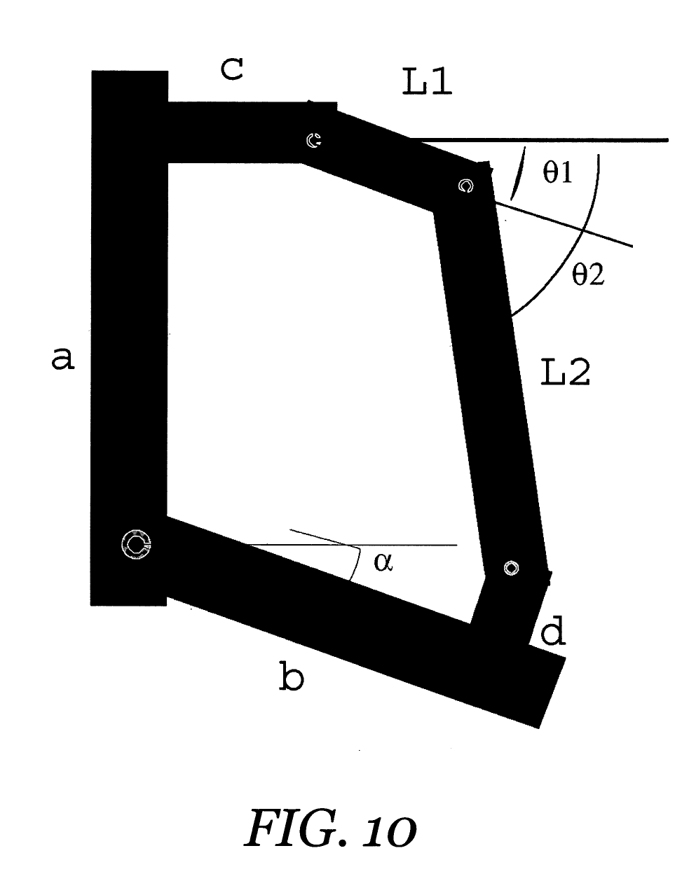

FIG. 10 shows a sagittal plane linkage diagram of the kinematic mechanism shown in FIG. 8. This is similar to a four-bar linkage with the leg, foot, links, and rods being the four links.

FIG. 11 depicts one exemplary embodiment of a leg connection. The leg connection can include a portion that contacts the leg, such as a piece with a curved contour, and a bracket that can support the transmission system.

FIG. 11A shows another exemplary embodiment of a leg connection that includes a knee brace. The knee brace may include a shin mount, a knee joint, and a thigh mount. In some embodiments, the brace can further include straps or the like that connect to waist to provide additional support.

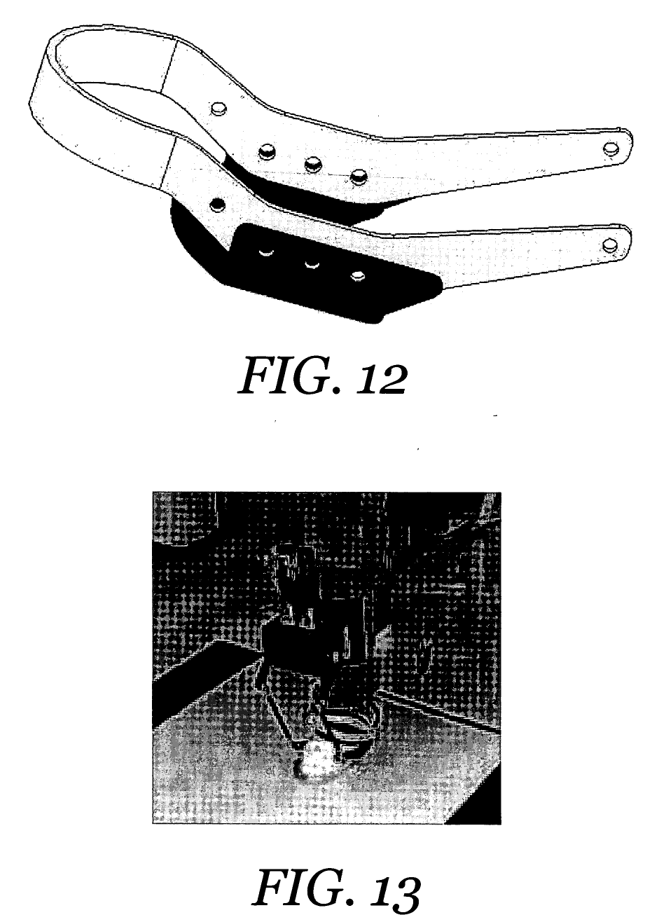

FIG. 12 depicts one exemplary embodiment of a foot connection. The foot connection can include a flanking piece connected to a supporting piece. The supporting piece receives the foot. The foot can be secured with a restraint, such as a strap. The flanking piece is disposed on either side of the foot. Rods connected to the links of the transmission system can couple to the flanking piece on either side of the foot, where the torques can be applied.

The leg and/or foot connections can also include one or more air bags, cushions, or other space-occupying objects to improve the fit and comfort of the ankle interface on patients of various sizes.

FIG. 13 is a photograph showing an ankle interface according to FIG. 2 installed on a subject's lower extremity.

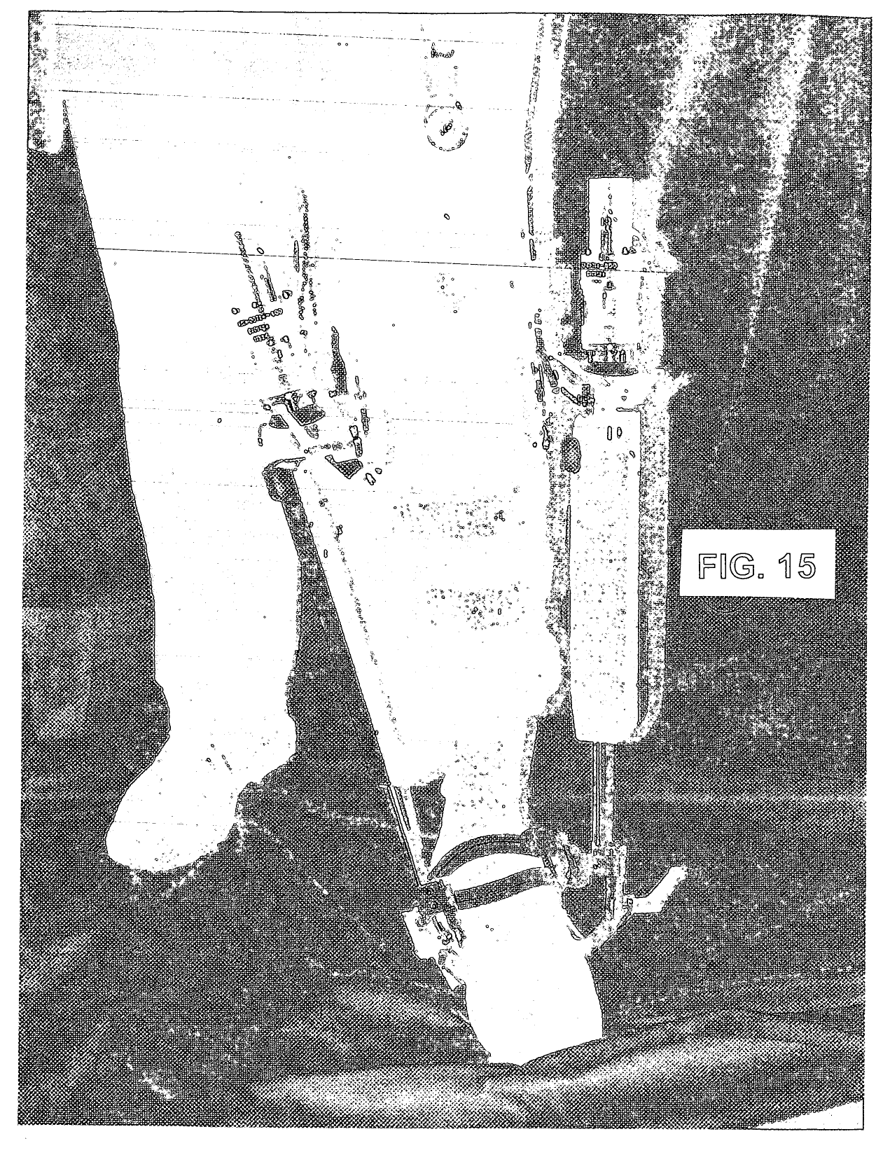

FIG. 15 is a photograph of another ankle interface. This interface includes a leg connection in the form of a knee brace 110 having upper 112 and lower 114 portions that are coupled at swivels 118. The brace may be positioned so that the swivels are aligned anteroposteriorly and superiorinferiorly with the knee to facilitate normal knee flexion-extension. Two actuators 120, 130 as previously described are mounted to the lower portion of the knee brace and extend to a foot connection 140 as previously described. Each actuator may include a motor 132 to drive the actuator and a spherical joint 134 to provide three degrees of freedom between the leg connection and the actuator (two degrees of freedom provided by the spherical joint and one by the actuator). A strap 150 extended around the subject's opposite shoulder (not shown) may be attached to the leg connection, such as to the upper portion. The shoulder strap can decrease the sense of added weight the ankle interface can cause the subject and so can facilitate a subject's normal gait while wearing the ankle interface.

The actuators of the FIG. 15 embodiment are positioned to the sides of the knee and are aligned in the same anteroposterior plane as (or as close as possible to) the knee's flexion/extension axis. Such positioning can decrease the inertial effects caused by rotation of the actuators around the knee. However, such positioning can cause the medial actuator 120 to hit against the subject's other leg and to occupy the space normally occupied by the subject's other knee, thereby disturbing the subject's gait and causing discomfort. This tendency to interfere with gait and knee position can be reduced by shortening the portion of the actuator extending above the knee.

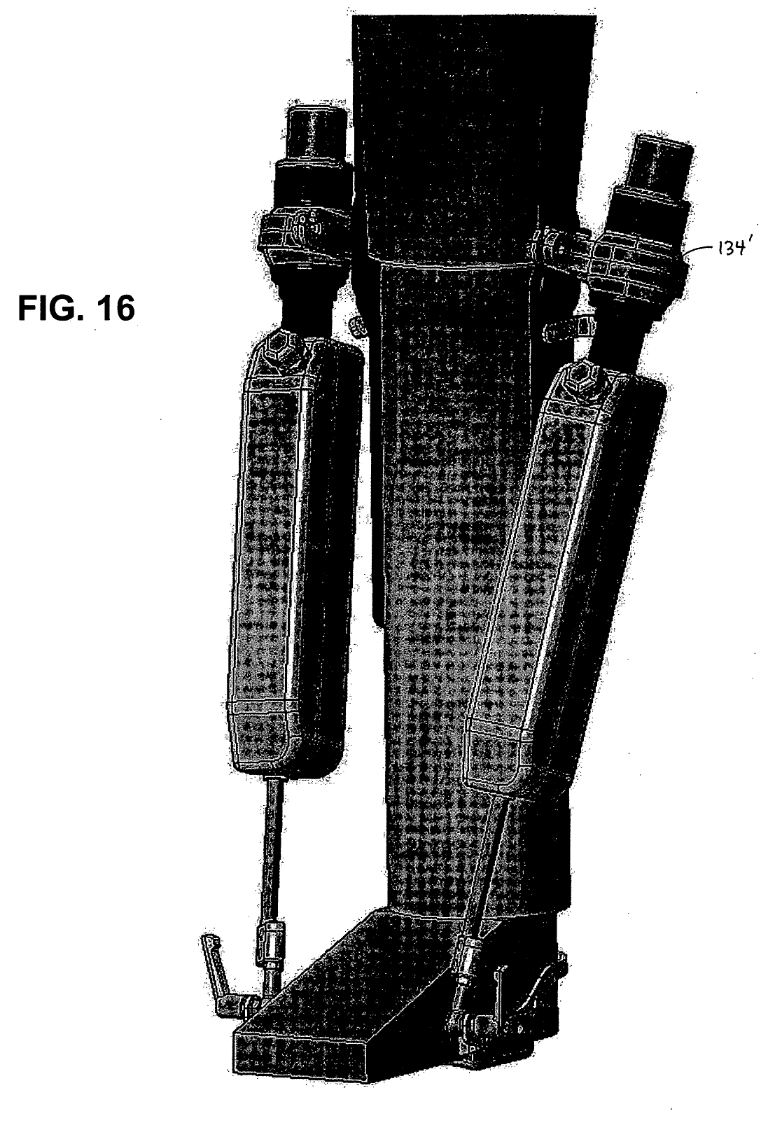

FIG. 16 depicts an embodiment of an ankle interface in which the length of the actuator above the knee is reduced by embedding the actuator's motor within the spherical joint. Spherical joint 134′ defines an internal cavity (not shown) to accommodate the motor (not shown), thereby decreasing the length of the actuator extending above the knee. With proper dimensioning, an interface according to this embodiment can avoid hitting against the subject's other leg but may still interfere with the other knee's normal positioning.

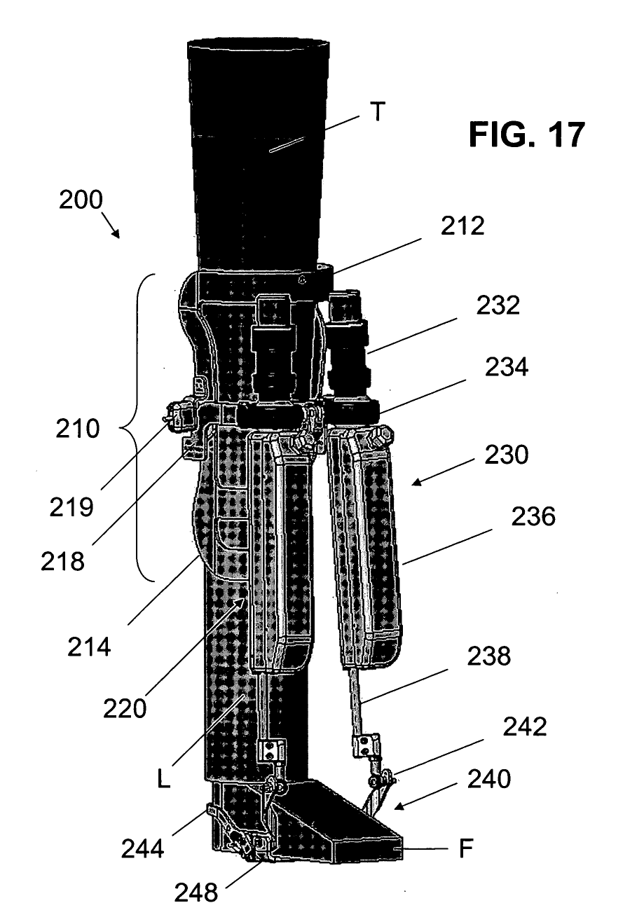

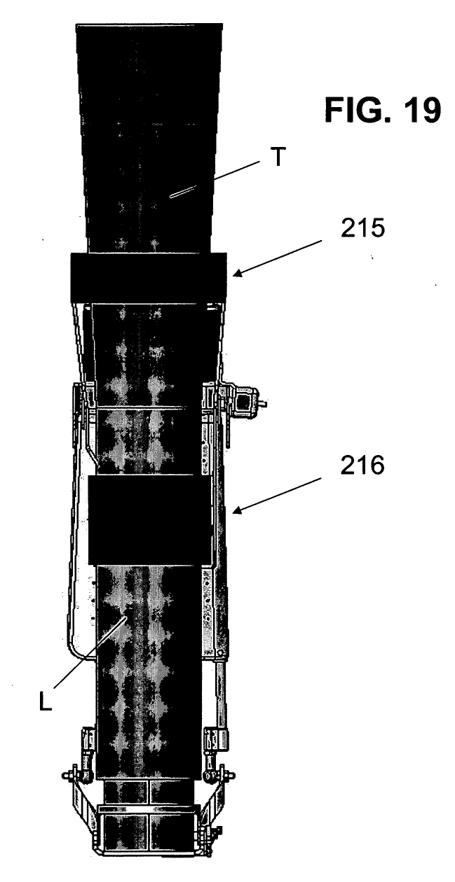

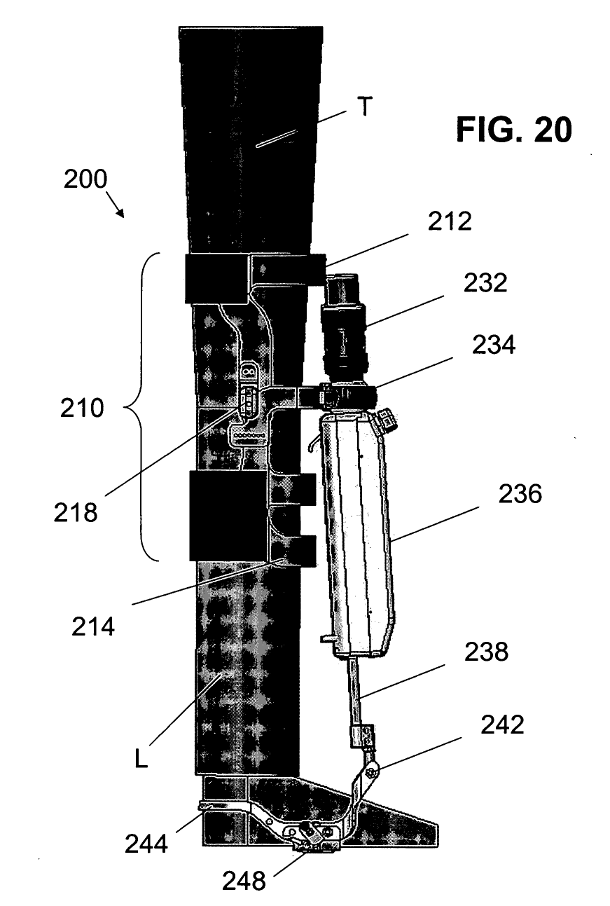

FIGS. 17-21 show various views of a further embodiment of an ankle interface in which the “knee knock” is reduced or eliminated by positioning the actuators slightly anterior to the knee's flexion/extension axis. Although such positioning reintroduces some inertial effects when the actuators rotate, it permits normal knee positioning and thus facilitates normal gait. The amount of anterior displacement is a function of the mass of the interface, the size of the subject, the percentage of muscle strength required to counteract the torque created upon movement of the anteriorly displaced actuators, and other factors. Depending on these variables, the anterior displacement should be no more than 5 centimeters, 4 centimeters, 3 centimeters, 2 centimeters, or 1 centimeter anterior to the knee flexion/extension axis. In some settings, it may be preferred that the anterior displacement be sufficiently small that the muscle strength percentage be at or below about 7% (the “just noticeable difference,” or “jnd” for this sensory input).

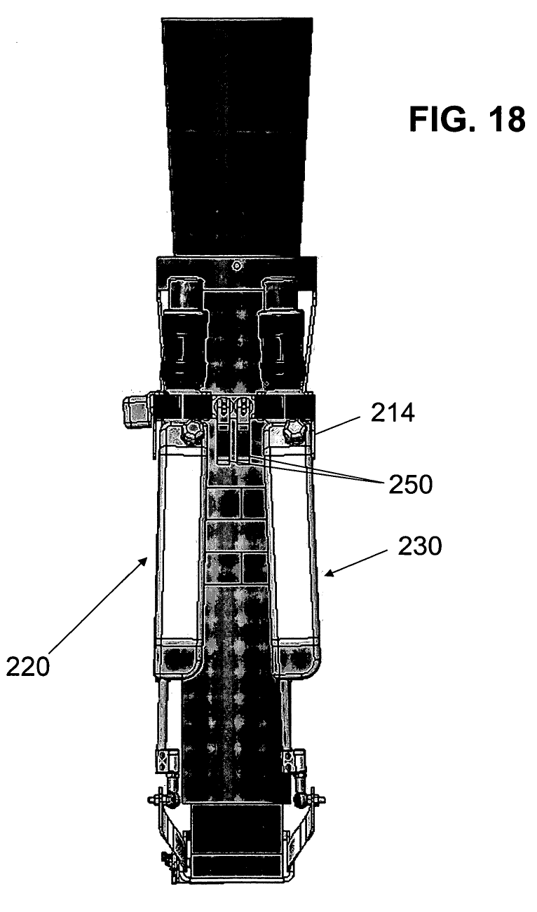

FIG. 17 provides an isometric view of this embodiment of an ankle interface; FIGS. 18-21 provide front, back, side, and bottom views of the same embodiment. As with the embodiments shown in FIGS. 15-16, the ankle interface 200 includes knee brace 210 forming the leg connection, with upper portion 212 and lower portion 214 attached at hinged joints 218 that line up on the axis of knee flexion/extension. The upper and lower portions of the knee brace may include straps 215, 216 that wrap around the subject's thigh T and lower leg L to help secure the interface to the subject. The upper portion of the knee brace may also include an attachment for receiving a shoulder strap, as discussed previously. The device may include one or more sensors, as described previously, such as knee angle position sensor 219.

In the depicted embodiment, actuators 220, 230 are coupled to the lower portion of the knee brace by spherical joints 234 to permit ankle motion with three degrees of freedom (dorsi/plantar flexion, inversion/eversion, and adduction/abduction). The actuators are, for example, traction screw drives 236 powered by motors 232. The drives cause rods 238 to advance and retract.

The distal ends of the rods are coupled to opposite ends of a foot connection 240 by way of joints 242. As discussed previously, the foot connection may include a flanking piece 244 that has roughly a U shape and extends around the back and sides of the foot, and a supporting piece 248 that crosses under the foot. A strap (not shown) may extend over the top of the foot in some embodiments. The supporting piece is positioned to cross under the foot some distance away from the ankle, so that forces exerted by the supporting piece upon the foot create torques on the ankle.

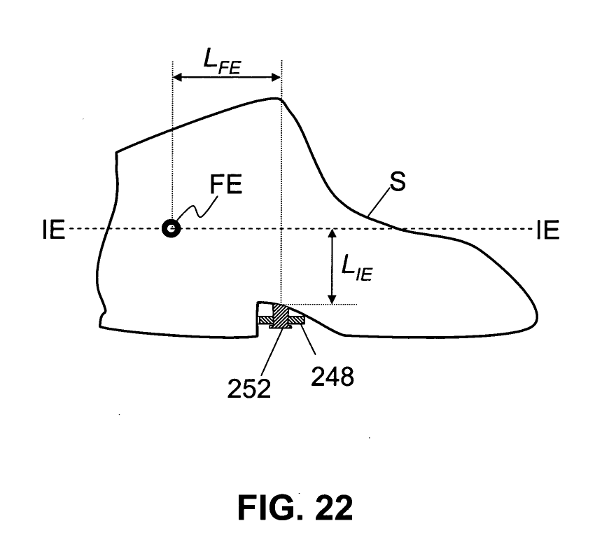

In the depicted embodiment, the supporting piece is positioned to run under the arch-supporting portion (sometimes called the “shank”) of a subject's shoe. Such positioning facilitates torque generation and also provides clearance for the connecting portion to contact and support the shoe while still allowing the shoe's sole and heel to touch the walking surface. FIG. 22 shows (in cross section) an exemplary position for supporting piece 248 relative to shoe S. While not to scale, this drawing demonstrates that when the connecting portion is so positioned, it is at distance L_{FE} from flexion-extension axis FE and distance L_{IE} from inversion-eversion axis IE. Consequently, forces transmitted from the connecting portion to the foot act at these distances from the relevant ankle axes and so cause torques upon the ankle.

As discussed previously, moving the two rods of the actuators in the same direction—that is, retracting them or advancing them together—applies a moment to the ankle to cause dorsi- or plantar flexion. Moving the two rods in opposite directions—advancing one while retracting the other—will exert a moment on the ankle to cause inversion or eversion. Although this embodiment does not actuate adduction/abduction, spherical joints 234 permit adduction/abduction so that the ankle retains the usual freedom of motion.

An ankle interface may also include various attachment points for assembling the device and attaching it to a subject. As shown in FIG. 18, actuators 220, 230 may be attached to the lower portion 214 of the knee brace by locks 250. These locks may have latches that allow for rapid opening and closing, so that the interface may be easily installed and removed to minimize preparation time. Including the locks in the interface can improve reproducibility of device positioning, because the operator does not have to judge where, for example, to position the connecting portion; instead, it simply snaps into place.

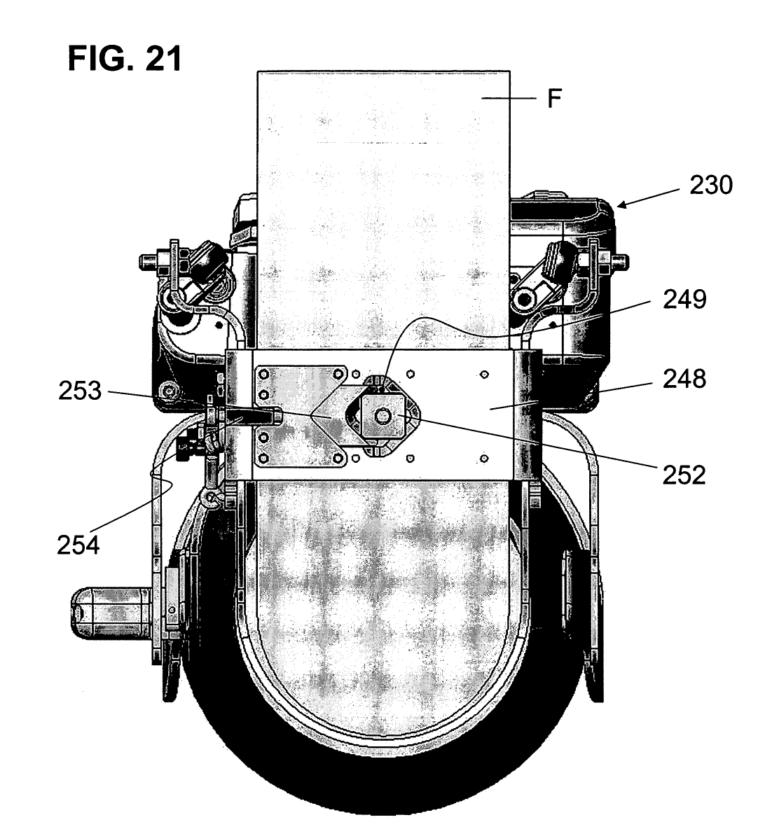

FIGS. 21 and 22 show another use of locks, in which the subject's shoe S includes cleat 252 protruding from the bottom of the shoe. The cleat protrudes through aperture 249 of supporting piece 248 when the subject's foot is positioned in the foot connection. Tongue 253 may then be tightened against the cleat by advancing bolt 254. The bolt may include a ratchet mechanism that prevents it from loosening during use.

A wide variety of attachment/release mechanisms may be used. In some embodiments, a subject's shoe may include a lock portion as described previously. The lock portion may be so sized and shaped as to fit, in a first orientation, through an aperture in the connection portion of the supporting piece of the foot connection and then can be transitioned to a second orientation in which it cannot pass back through the aperture.

One exemplary process for installing the device on a subject for use includes:
a. placing the knee brace on the subject's knee and securing the straps;
b. having the subject put on a shoe with a locking portion installed in the shank;
c. locking the shoe onto the connection portion of the foot connection; and
d. locking the interface to the lower portion of the knee brace.

Ankle interfaces built as described herein can provide one or more benefits:

The device can be lightweight, so that it does not burden the patient.

The weight can placed close to the knee to minimize inertial effects.

The device can be combined with other modules (e.g. pelvis, hip, knee) or used independently.

It can be used on a treadmill or over ground.

It can be installed on either leg.

### EXAMPLE

This example is provided for illustrative purposes to describe one particular embodiment of an ankle interface. It is not intended to be limiting.

Two Kollmorgen RBE(H) 00714 actuators were used to produce a maximum continuous torque of 0.50 N-m (0.25 N-m each), and were augmented by 30:1 gear reduction. A Bayside PS 40-010 planetary gearhead with a ratio of 10:1 was mounted inline with each motor. An additional reduction of 3:1 was supplied with bevel gears, which also serves to change the axis of the applied torque. Additional torque amplification of approximately 1.5:1 was achieved in dorsi/plantar flexion from mechanical advantage in the mechanism. This resulted in a net torque of approximately 23 N·m in dorsi/plantar flexion and 15 N·m in inversion/eversion. The gears and upper links rotated on a crossed-roller bearing (THK RB 2008), which can withstand the axial and moment loads produced by the rotating gears. The upper links connected to the lower links with spherical joint rod ends (THK AL 6D). Rod ends also connected these lower links to the foot connection piece. Position (and velocity) information was provided by Gurley R19 encoders mounted co-axial with the motors and torques measured by a torque sensor.

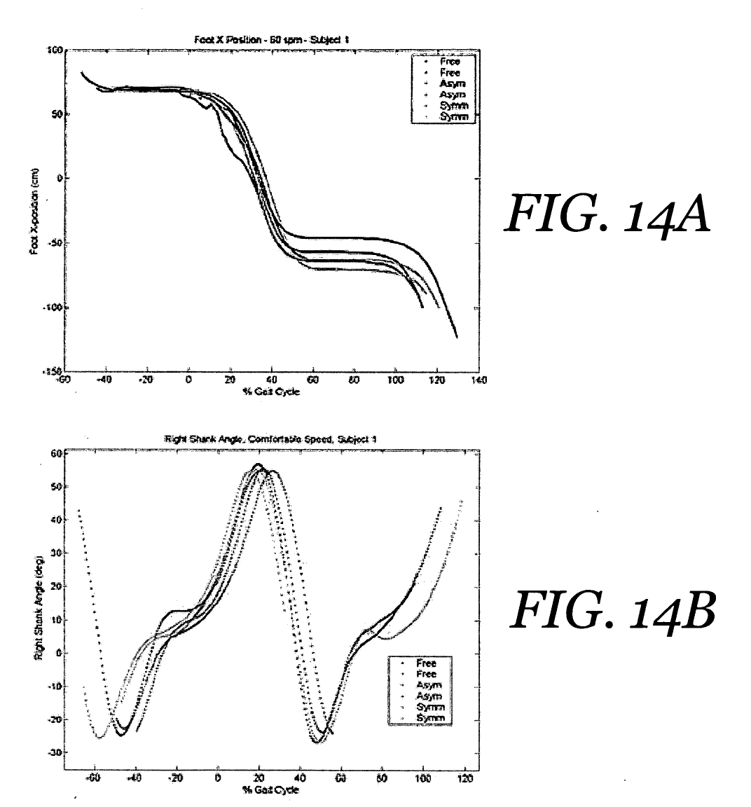

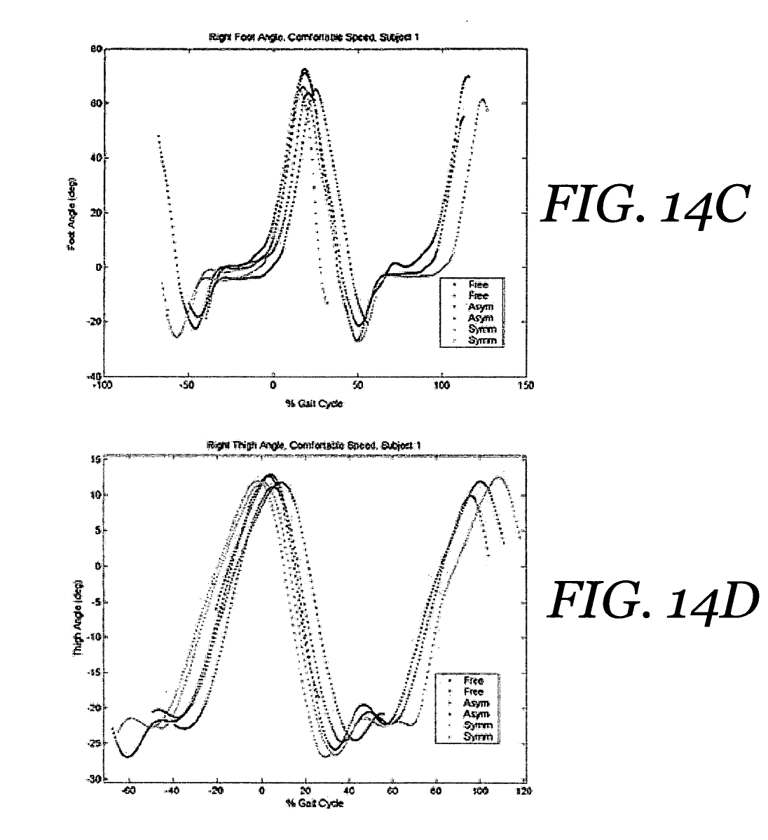

The patient's foot (with shoe on) was secured to this piece with a single strap over the hind foot. The foot connection piece does not extend the entire length of the patient's shoe but is designed to end near the midtarsals, to allow forefoot mobility. FIG. 14A-D show the kinematics of this embodiment with unimpaired subjects comparing three different walking conditions: a) “free walking”, b) walking with asymmetric loading (ankle module on one leg), and c) walking with symmetric loading (ankle module and dummy load on each leg).

1. An ankle interface, comprising:
a leg connection attachable to a user's leg;
a foot connection attachable to the user's corresponding foot; and
a transmission system coupling the leg connection and the foot connection with at least two degrees of freedom and actuating at least two degrees of freedom.

2. The ankle interface of claim 1, wherein the transmission system comprises at least one motor providing actuation.

3. The ankle interface of claim 1, wherein the transmission system comprises two motors, and two link mechanisms in parallel to one another, each link mechanism coupled on the proximal end to the respective motor and on the distal end to respective sides of the foot connection.

4. The ankle interface of claim 3, wherein each link mechanism comprises a linear friction actuator.

5. The ankle interface of claim 3, wherein each link mechanism comprises a traction drive actuator.

6. The ankle interface of claim 1, wherein the transmission system couples the leg connection and the foot connection with three degrees of freedom.

7. The ankle interface of claim 6, wherein the transmission system so actuates the foot connection as to actuate an ankle flexion/extension degree of freedom and an ankle inversion/eversion degree of freedom.

8. The ankle interface of claim 1, further comprising a shoulder strap.

9. The ankle interface of claim 1, wherein the transmission system actuates the foot connection in three degrees of freedom.

10. The ankle interface of claim 1, wherein the transmission system further comprises at least one sensor producing an output indicative of a state of the ankle interface.

11. The ankle interface of claim 10, wherein the sensor comprises a position sensor.

12. The ankle interface of claim 10, wherein the sensor comprises a torque sensor.

13. The ankle interface of claim 1, wherein the leg connection comprises a knee brace, the knee brace including an upper portion coupled to a lower portion by at least one hinge joint.

14. The ankle interface of claim 13, wherein the transmission system is coupled to the knee brace lower portion.

15. The ankle interface of claim 1, wherein the foot connection comprises a flanking piece having a back portion sized and shaped to fit around the back of a subject's foot and two side portions sized and shaped to fit along the sides of the subject's foot, and a supporting piece spanning the two side portions.

16. The ankle interface of claim 1, wherein the transmission system is reversibly coupled to the leg connection by a locking system.

17. The ankle interface of claim 1, wherein the transmission system is reversibly coupled to the foot connection by a locking system.

18. The ankle interface of claim 17, wherein:
the foot connection comprises a flanking piece having a back portion sized and shaped to fit around the back of a subject's foot and two side portions sized and shaped to fit along the sides of the subject's foot, and a supporting piece spanning the two side portions and defining an aperture; and
the locking system comprises a cleat attached to a subject's shoe, the cleat being transitionable between a first state, in which the cleat is so oriented as to pass through the aperture, and a second state, in which the cleat is so oriented as not to pass through the aperture.

19. An ankle interface, comprising:
a leg connection including a knee brace having an upper portion coupled to a lower portion by at least one hinge joint;
a foot connection including a flanking piece having a back portion sized and shaped to fit around the back of a subject's foot and two side portions sized and shaped to fit along respective sides of the subject's foot, and a supporting piece spanning the two side portions; and
a transmission system coupling the leg connection and the foot connection with at least two actuated degrees of freedom, the transmission system including two link mechanisms, each link mechanism coupled at its proximal end to the knee brace lower portion and at its distal end to one of the foot connection side portions, and each link mechanism coupled to a motor.

20. An ankle motion system, comprising:
the ankle interface of claim 1; and
a controller coupled to the transmission system to control the actuation of the transmission system.

21. The ankle motion system of claim 20, further comprising at least one sensor coupled to the transmission system and producing an output indicative of a state of the ankle interface, wherein the controller controls actuation of the transmission system in response to the sensor output.

22. A method of ankle training, comprising:
attaching a subject's leg and foot to an ankle interface as defined in claim 1; and
actuating the transmission system to provide at least one of assistance, perturbation, and resistance to an ankle motion.

23. The method of claim 22, wherein the ankle motion comprises flexion and/or extension.

24. The method of claim 22, wherein the ankle motion comprises inversion and/or eversion.

25. A method of ankle training, comprising:
securing the knee brace of the ankle interface of claim 19 to a subject's knee;
having the subject put on a shoe with a cleat installed in a shank of the shoe;
locking the shoe onto the supporting piece of the foot connection; and
locking the interface to the lower portion of the knee brace.
